(19) United States
(12) Patent Application Publication
(10) Pub. No.: US 2026/0309972 A1
(43) Pub. Date: Oct. 8, 2026

(54) MICROSCOPE WITH DIGITAL BINOCULAR ASSEMBLY HAVING RIGID FLEX CABLE CONNECTIONS

(71) Applicant: Alcon Inc., Fribourg (CH)

(72) Inventors: Carlos Vasseur, Huntington Beach, CA (US); Mauricio Jochinsen, Laguna Beach, CA (US); Yuchao Liu, Irvine, CA (US)

(21) Appl. No.: 19/637,424

(22) Filed: Apr. 2, 2026

Related U.S. Application Data
(60) Provisional application No. 63/784,490, filed on Apr. 7, 2025.

(51) Int. Cl. G02B 21/36; G02B 21/00; G02B 21/20

(52) U.S. Cl. CPC G02B 21/368; G02B 21/0028; G02B 21/20; G02B 21/361; G02B 21/362; G02B 21/367

(57) ABSTRACT

Digital binoculars for use with an ophthalmic microscope include a pair of eyepieces, a housing connectable to the ophthalmic microscope and to the pair of eyepieces, a pair of micro-display screens disposed within the housing cavity, and a printed circuit board assembly (PCBA) connected to the housing, in communication with the ophthalmic microscope, and configured to control operation of the micro-display screens. A pair of rigid flex cables connect a respective one of the micro-display screens to the PCBA. An interpupillary distance (IPD) adjustment mechanism is connected to the housing and configured to translate the pair of eyepieces towards or away from one another to thereby adjust an IPD of a user of the digital binoculars within a predetermined IPD range. The rigid flex cables accommodate translation of the micro-display screens relative to the PCBA during an adjustment of the IPD.

### INTRODUCTION

The present disclosure relates generally to a digital binocular assembly for use with an ophthalmic microscope.

During ophthalmic surgery, e.g., a cataract or vitreoretinal surgery, a patient is placed on a surgical platform beneath a microscope. A surgeon uses the microscope to view the patient’s ocular anatomy under high levels magnification. A digital camera may be integrated with the microscope to provide imaging capabilities, with the microscope also having a microscope lamp for illuminating the eye. Internal illumination may be provided by an endoilluminator or a chandelier that the surgeon inserts into the eye’s vitreous chamber. Using oculars attached to the microscope, and possibly a heads-up display (HUD) or high-resolution monitors, the surgeon and supporting medical staff are able to view projections of the patient’s eye in real-time during the surgery, as well as during in-office visits performed before or after the surgery.

### SUMMARY

Disclosed herein are digital binoculars for use with an ophthalmic microscope. The binoculars include right and left eyepieces allowing a user to view a target eye via separate right and left channels, and thus in stereo. The binoculars are equipped with an interpupillary distance (IPD) adjustment mechanism that accommodates a defined IPD range, e.g., about 55 millimeters (mm) to about 65mm in a possible implementation. The binoculars may include a multi-axis adjustment mechanism operable for aligning internal micro-displays of the binoculars, typically during manufacturing of the binoculars. To enable lateral motion needed to accommodate adjustment to the full IPD range, the binoculars are equipped with rigid flex cables that connect the micro-displays to a main printed circuit board assembly (PCBA) serving as the microscope’s main control board.

In accordance with a representative embodiment, the digital binoculars include a pair of eyepieces, a housing, and a pair of micro-displays connectable to the microscope and to the eyepieces. The housing defines a housing cavity. The micro-display screens are disposed within the housing cavity. The above-noted PCBA is connected to the housing and in communication with the microscope, with the PCBA being configured to control operation of the micro-display screens and other functions of the microscope.

A pair of rigid flex cables connect a respective one of the micro-display screens to the PCBA in this implementation. An IPD adjustment mechanism connected to the housing is used to translate the eyepieces toward or away from one another to thereby adjust an IPD of a user of the digital binoculars within the defined IPD range. Thus, the rigid flex cables are configured to accommodate motion of the micro-display screens relative to the PCBA during adjustment of the IPD adjustment mechanism.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 illustrates an operating room setup with an ophthalmic microscope having digital binoculars that are constructed as set forth herein.

FIG. 2 illustrates a perspective view of the representative digital binoculars of FIG. 1.

FIG. 3 illustrates a side view illustration of digital binoculars in accordance with a possible construction.

FIG. 4 illustrates a cutaway top view illustration of the digital binoculars of FIG. 3 showing an interpupillary distance (IPD) adjustment mechanism.

FIG. 5 illustrates a side schematic view of a portion of the digital binoculars shown in FIG. 4 showing a rigid flex cable connection.

FIG. 6 is a cutaway rear view illustration of the digital binoculars of FIGS. 2-5.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Embodiments of the present disclosure are described in detail herein. Disclosed embodiments are provided as examples, with other embodiments possibly taking alternative forms. The Figures are not necessarily drawn to scale. For instance, some features could be exaggerated or minimized to show details of particular components. Therefore, specific structural and functional details disclosed herein are not to be interpreted as limiting, but merely as a representative basis for teaching one skilled in the art to employ the present disclosure.

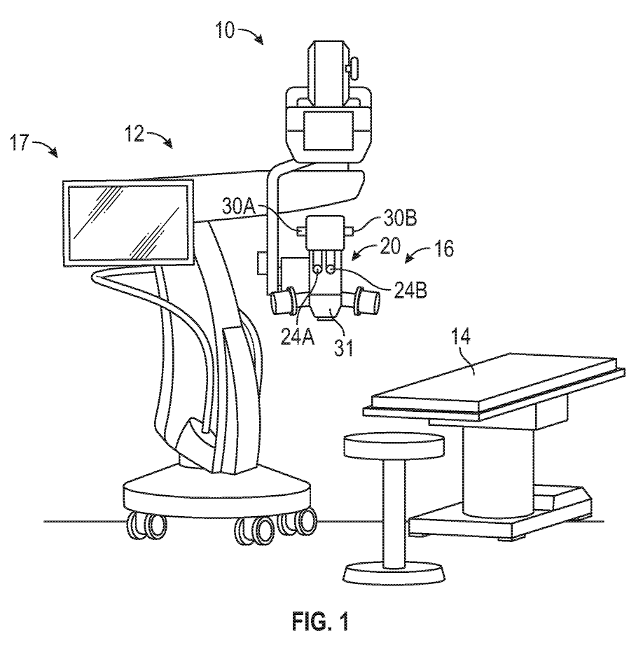

Referring to the drawings, wherein like reference numbers refer to like components, an ophthalmic suite 10 is depicted schematically in FIG. 1. The range of possible ophthalmic procedures that could be performed within the ophthalmic surgical suite 10 include, e.g., vitreoretinal surgery, cataract surgery, visualization, etc. As appreciated by those skilled in the art, the ophthalmic suite 10 of FIG. 1 may be equipped with a multi-axis support arm 12 and an operating platform 14. The support arm 12, which may be part of a multi-axis robot in some embodiments, a non-robotic serial arm, or another construction, is typically connected to an ophthalmic microscope 16 through which a surgeon (not shown) is able to view a surgical field. For example, using associated hardware and software, the surgeon is able to view magnified real-time images of a surgical field of view on a display screen 17, several of which may be present within the ophthalmic suite 10.

Various embodiments of the microscope 16 may be used within the scope of the disclosure, for instance the LuxOR^{®} Revalia^{™} or another application-suitable alternative. The microscope 16 may be used to provide variable levels of magnification for visualizing ocular anatomy, which in turn allows the surgeon to precisely identify tissues, blood vessels, lenses, vitreous humor, and other portions of a patient’s eye. In addition to magnification, the microscope 16 may also include a lamp 31 providing external illumination of a surgical site. Although omitted from FIG. 1 for illustrative simplicity, the microscope 16 may be in communication with a surgical console operable for energizing and controlling one or more surgical tools used in the course of surgery, e.g., an endoilluminator, a vitrector, a phacoemulsifier tool, capsulorhexis forceps, etc. With each tool used in a given stage or stages of a given cataract or vitreoretinal surgery, the surgical console may be operable for communicating the particular surgical stage to the microscope 16 for control thereof.

The microscope 16 includes or is connected to a digital binoculars 20 constructed as set forth below with reference to FIGS. 2-6, i.e., with separate first (left) and second (right) eyepieces 24A and 24B, respectively, for stereoscopic vision. Together, the microscope 16 and the digital binoculars 20 form an ophthalmic microscope assembly 200. Handles 30A and 30B are attached to the microscope 16 to allow the surgeon to adjust an interpupillary distance (IPD), i.e., a linear distance between respective optical axes of the first and second eyepieces 24A, 24B, to the surgeon’s preference as set forth below.

As appreciated, digital oculars or eyepieces are used to enhance visualization and ergonomics during the above-noted procedures when using the microscope 16. The digital binoculars 20 may be used to adjust magnification levels in conjunction with magnification by the objective lens of the microscope 16, along with diopter correction, e.g., via diopter adjustment rings 32A and 32B (FIG. 2) to help compensate for a surgeon’s refractive error. Therefore, each of the eyepieces 24A and 24B may include a corresponding diopter adjustment ring 32A or 32B, the rotation of which is configured to adjust a diopter level of a respective one of the pair of eyepieces 24A and 24B.

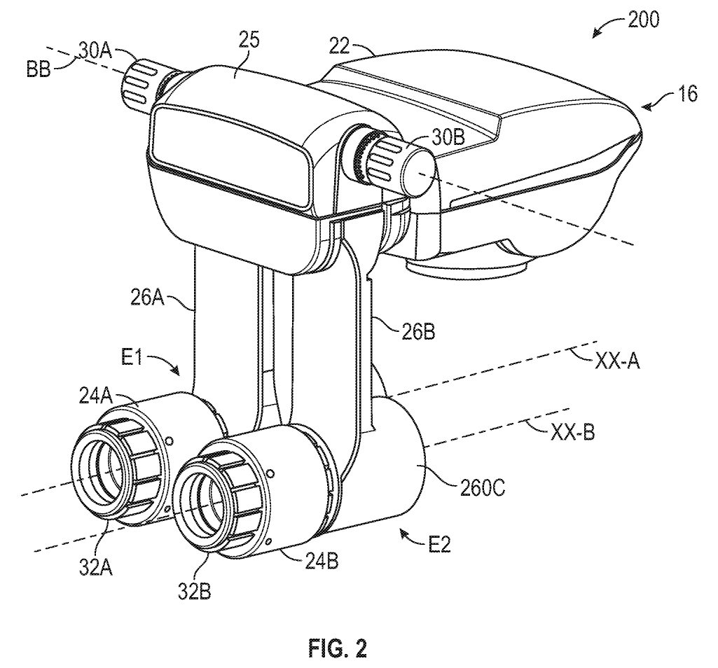

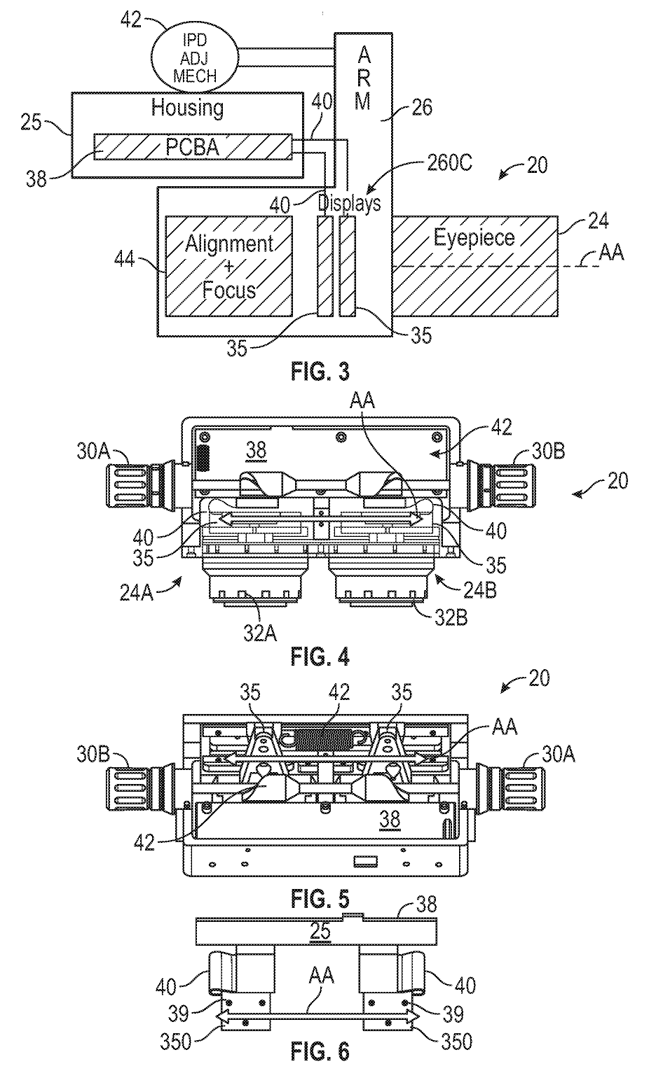

Referring to FIG. 2, in a non-limiting representative construction of the digital binoculars 20, the digital binoculars 20 may include a housing 25 having first and second axial arms 26A and 26B connected to or positioned adjacent to the handles 30A and 30B. A separate housing 22 of the microscope 16 may contain other components and lenses of the microscope 16. The respective first and second arms 26A and 26B extend vertically downward from the housing 22 to support and interconnect the housing 22 and to the first and second eyepieces 24A and 24B, respectively. The first and second eyepieces 24A and 24B in turn are attached to distal ends E1 and E2 of the first and second arms 26A and 26B to allow the surgeon to view the surgical field at a reduced height. A beneficial feature of having the first and second eyepieces 24A and 24B located at the distal ends E1 and E2 of the first and second arms 26A and 26B is improved comfort when performing a procedure that would otherwise require the microscope 16 to be positioned at a height that may be uncomfortable to reach by some surgeons. A possible embodiment of the digital binoculars 20 will now be described with reference to FIGS. 3-6.

FIG. 3 is a side view illustration of the digital binoculars 20 in accordance with a representative construction. One of the pair of eyepieces 24A or 24B of FIG. 2, shown generically as eyepiece 24, is connected to a corresponding one of the first or second arms 26A, 26B, shown generically as arm 26. A pair of micro-display screens 35 may be packaged within a cavity 260C defined by the arm 26, along with an alignment and focus mechanism 44 disposed within the cavity 260C, e.g., a set of screws, pins, or other adjustment pieces that can be manipulated during manufacturing of the digital binoculars 20 to calibrate or adjust an alignment setting and focus setting of the digital binoculars 20. The arm 26 is connectable to the microscope 16 of FIG. 1 or formed integral therewith, with the arm 26 also connected to the pair of eyepiece 24, i.e., the eyepieces 24A and 24B of FIG. 2.

As part of the present construction, the micro-display screens 35 are disposed within the cavity 260C. Each of the micro-display screens 35 is aligned with a corresponding optical axis XX-A or XX-B of a corresponding one of the eyepieces 24A and 24B of FIG. 2. Each eyepiece 24A or 24B thus has a corresponding optical axis XX-A, XX-B arranged orthogonal to a common center axis (BB) of the handles 30A, 30B. The micro-display screens 35 may be variously embodied as light-emitting diode (LED) screens, organic light-emitting diode (OLED) screens, or liquid crystal on silicon (LCoS) screens in different implementations.

A printed circuit board assembly (PCBA) 38 is connected to and/or contained within the housing 25 (FIG. 2), and thus is in wired or wireless communication with the microscope 16 of FIG. 1. The PCBA 38 as contemplated herein is configured to control operation of the micro-display screens 35 and possibly other functions of the microscope 16 and digital binoculars 20. An interpupillary distance (IPD) adjustment mechanism 42 is connected to the housing 25 and configured to translate the eyepieces 24A, 24B of FIG. 2 towards or away from one another to thereby adjust to an IPD of a user of the digital binoculars 20 within a predetermined IPD range. Representative placement of the IPD adjustment mechanism 42 is illustrated in FIGS. 4-6 and described in further detail below.

RIGID FLEX PCBs: The digital binoculars 20 also include a pair of rigid flex PCBs 40 configured to accommodate translation of the micro-display screens 35 relative to the PCBA 38 during an adjustment of the IPD. Each rigid flex PCB 40 connects a respective one of the micro-display screens 35 to the PCBA 38. As appreciated in the art, a rigid flex PCB is a hybrid circuit board integrating rigid PCB sections of FR-4 or another suitable material with flexible, copper trace-containing circuit sections, e.g., polyimide, to form a flexible circuit connection. The digital binoculars 20 employ the rigid flex PCBs 40 herein to accommodate relative motion of the micro-display screens 35 relative to the PCBA 38 during an adjustment of the IPD adjustment mechanism 42.

Referring to FIG. 4, which shows internal detail of an embodiment of the digital binoculars 20 from above (with a portion of the housing 25 removed for clarity), the handles 30A, 30B are disposed at distal ends of the IPD adjustment mechanism 42 along a common center axis. Rotation of the handles 30A, 30B together in a first direction, i.e., clockwise or counterclockwise, translates the micro-display screens 35 away from each other to increase the IPD within a predetermined IPD range, as indicated by double-headed arrow AA. Conversely, rotation of the handles 30A, 30B in a second direction opposite the first direction decreases the IPD within the predetermined IPD range. The IPD adjustment mechanism 42 is adjustable herein to accommodate the full average IPD range for adults, for instance about 55-65 millimeters (mm), or about 55-75 mm in other implementations.

A possible embodiment of the IPD adjustment mechanism 42 is shown in FIG. 5, which illustrates the digital binoculars 20 of FIG. 4 as one looks toward the eyepieces 24A, 24B of FIG. 4, the IPD adjustment mechanism 42 may be constructed in numerous ways. For instance, the IPD adjustment mechanism 42 may be constructed as a spring-biased or geared mechanism that, when rotated in a first direction by manual torque to the handles 30A, 30B, forces the micro-displays 35 away from each other. Opposing translational direction may continue until the micro-displays 35 are at a maximum distance within the predetermined IPD range, or about 65 mm to about 75mm in a representative construction. Similarly, rotation of the handles 30A and 30B in the opposite direction will cause the micro-displays 35 to move closer together. This movement continues until the micro-displays 35 are at a minimum distance within the predetermined IPD range, or about 55 mm in the above-noted representative construction

Referring briefly to FIG. 6, the micro-display screens 35 of FIGS. 3 and 4 may be mounted to a display support bracket 350, e.g., via mounting holes 39. The rigid flex PCBs 40, once secured to the support brackets 350, may be connected to the PCBA 38 via the rigid flex PCBs 40. Thus, as a user rotates the handles 30A, 30B of FIG. 4 to adjust the IPD, the resulting translation of the micro-display screens 35 towards or away from one another is compensated for by the resilience/flexibility of the rigid flex PCBs 40. Among other attendant benefits, the inclusion of the rigid flex PCBs 40 allows for construction of the digital binoculars 20 in a host of different configurations. Additionally, use of the rigid flex PCBs 40 facilitates operation of the alignment and focus mechanism 44 (FIG. 3), in particular relative motion of the micro-displays 35 and the eyepieces 24A, 24B when calibrating the digital binoculars 20. These and other benefits will be readily appreciated by those skilled in the art now having the benefit of the present teachings.

For purposes of this Detailed Description, unless specifically disclaimed: the singular includes the plural and vice versa; the words “and” and “or” shall be both conjunctive and disjunctive; the words “any” and “all” shall both mean “any and all”; and the words “including,” “containing,” “comprising,” “having,” and the like, shall each mean “including without limitation.” Moreover, words of approximation, such as “about,” “almost,” “substantially,” “generally,” “approximately,” and the like, may each be used herein to denote “at, near, or nearly at,” or “within 0-5% of,” or “within acceptable manufacturing tolerances,” or any logical combination thereof, for example. Lastly, directional adjectives and adverbs, such as fore, aft, inboard, outboard, starboard, port, vertical, horizontal, upward, downward, front, back, left, right, etc., may be with respect to a user standing on a horizontal surface.

While various embodiments have been described, the description is intended to be exemplary rather than limiting. It will be apparent to those of ordinary skill in the art that many more embodiments and implementations are possible that are within the scope of the embodiments. Any feature of any embodiment may be used in combination with or substituted for any other feature or element in any other embodiment unless specifically restricted. Accordingly, the embodiments are not to be restricted except in light of the attached claims and their equivalents. Also, various modifications and changes may be made within the scope of the attached claims.

What is claimed is:

1. Digital binoculars for use with an ophthalmic microscope, comprising:
a pair of eyepieces;
a housing connectable to the ophthalmic microscope and to the pair of eyepieces, the housing defining a housing cavity;
a pair of micro-display screens disposed within the housing cavity;
a printed circuit board assembly (PCBA) connected to the housing, in communication with the ophthalmic microscope, and configured to control operation of the micro-display screens;
a pair of rigid flex cables each connecting a respective one of the micro-display screens to the PCBA; and
an interpupillary distance (IPD) adjustment mechanism connected to the housing and configured to translate the pair of eyepieces towards or away from one another to thereby adjust an IPD of a user of the digital binoculars within a predetermined IPD range, wherein the rigid flex cables are configured to accommodate translation of the micro-display screens relative to the PCBA during an adjustment of the IPD.

2. The digital binoculars of claim 1, wherein:
the IPD adjustment mechanism includes handles disposed at distal ends of the IPD adjustment mechanism, and wherein a rotation of the handles in opposing directions increases and decreases the IPD.

3. The digital binoculars of claim 1, wherein the predetermined IPD range is about 55 millimeters (mm) to about 75mm.

4. The digital binoculars of claim 3, wherein the predetermined IPD range is about 65mm to about 75mm.

5. The digital binoculars of claim 1, further comprising:
an alignment and focus mechanism that is disposed within the housing cavity and configured to adjust an alignment setting and a focus setting of the digital binoculars.

6. The digital binoculars of claim 1, wherein each respective eyepiece of the pair of eyepieces includes a corresponding diopter adjustment ring configured to adjust a diopter level of the pair of eyepieces.

7. The digital binoculars of claim 1, wherein the micro-display screens include organic light-emitting diode (OLED) screens or liquid crystal on silicon (LCoS) screens.

8. The digital binoculars of claim 1, further comprising:
a pair of arms extending vertically between and interconnecting the housing and the eyepieces.

9. An ophthalmic microscope assembly, comprising:
an ophthalmic microscope; and
digital binoculars connected to the ophthalmic microscope, including:
a pair of eyepieces;
a housing connectable to the ophthalmic microscope and to the pair of eyepieces, the housing defining a housing cavity;
a pair of micro-display screens disposed within the housing cavity;
a printed circuit board assembly (PCBA) connected to the housing, in communication with the ophthalmic microscope, and configured to control operation of the micro-display screens;
a pair of rigid flex cables each connecting a respective one of the micro-display screens to the PCBA; and
an interpupillary distance (IPD) adjustment mechanism connected to the housing and configured to translate the pair of eyepieces towards or away from one another to thereby adjust an IPD of a user of the digital binoculars within a predetermined IPD range, wherein the rigid flex cables are configured to accommodate translation of the micro-display screens relative to the PCBA during an adjustment of the IPD.

10. The ophthalmic microscope assembly of claim 9, wherein:
the IPD adjustment mechanism includes handles disposed at distal ends of the IPD adjustment mechanism, and wherein a rotation of the handles in opposing directions increases and decreases the IPD.

11. The ophthalmic microscope assembly of claim 10, wherein each eyepiece of the pair of eyepieces has a corresponding optical axis arranged orthogonal to a common center axis of the handles.

12. The ophthalmic microscope assembly of claim 9, wherein the predetermined IPD range is about 55 millimeters (mm) to about 75mm.

13. The ophthalmic microscope assembly of claim 12, wherein the predetermined IPD range is about 65mm to about 75mm.

14. The ophthalmic microscope assembly of claim 12, further comprising:
an alignment and focus mechanism disposed within the housing cavity and configured to adjust an alignment setting and a focus setting of the digital binoculars.

15. The ophthalmic microscope assembly of claim 9, wherein each respective eyepiece of the pair of eyepieces includes a corresponding diopter adjustment ring configured to adjust a diopter level of the pair of eyepieces.

16. The ophthalmic microscope assembly of claim 9, wherein the micro-display screens include organic light-emitting diode (OLED) screens or liquid crystal on silicon (LCoS) screens.

17. The ophthalmic microscope assembly of claim 9, further comprising:
a pair of axial arms extending vertically between and interconnecting the housing and the eyepieces.

18. Digital binoculars for use with an ophthalmic microscope, comprising:
a pair of eyepieces, wherein each respective eyepiece of the pair of eyepieces includes a corresponding diopter adjustment ring configured to adjust a diopter level of the pair of eyepieces;
a housing connectable to the ophthalmic microscope and to the pair of eyepieces, the housing defining a housing cavity;
a pair of micro-display screens disposed within the housing cavity;
a printed circuit board assembly (PCBA) connected to the housing, in communication with the ophthalmic microscope, and configured to control operation of the micro-display screens;
a pair of rigid flex cables each connecting a respective one of the micro-display screens to the PCBA;
a pair of axial arms extending vertically between and interconnecting the housing and the eyepieces; and
an interpupillary distance (IPD) adjustment mechanism connected to the housing and configured to translate the pair of eyepieces towards or away from one another to thereby adjust an IPD of a user of the digital binoculars within a predetermined IPD range of about 55 millimeters (mm) to about 75mm, the IPD adjustment mechanism including handles disposed at distal ends of the IPD adjustment mechanism, wherein a rotation of the handles in opposing directions increases and decreases the IPD, and wherein the rigid flex cables are configured to accommodate translation of the micro-display screens relative to the PCBA during an adjustment of the IPD.

19. The digital binoculars of claim 18, further comprising:
an alignment and focus mechanism that is disposed within the housing cavity and configured to adjust an alignment setting and a focus setting of the digital binoculars.

20. The digital binoculars of claim 18, wherein the micro-display screens include organic light-emitting diode (OLED) screens or liquid crystal on silicon (LCoS) screens.
